(19) United States
(12) Patent Application Publication
(10) Pub. No.: US 2014/0206867 A1
(43) Pub. Date: Jul. 24, 2014

(54) Process for Preparing Cyclopentylamine Derivatives and Intermediates Thereof

(71) Applicants: Anil Shahaji Khile, Navi Mumbai (IN); Vignesh Nair, Kerala (IN); Nikhil Trivedi, Maharashtra (IN); Nitin Sharadchandra Pradhan, Maharashtra (IN)

(72) Inventors: Anil Shahaji Khile, Navi Mumbai (IN); Vignesh Nair, Kerala (IN); Nikhil Trivedi, Maharashtra (IN); Nitin Sharadchandra Pradhan, Maharashtra (IN)

(73) Assignee: Actavis Group PTC EHF, Hafnarfjorour (IS)

(21) Appl. No.: 14/126,102

(22) PCT Filed: Jun. 13, 2012
(86) PCT No.: PCT/IB2012/001344

§ 371 Date: Mar. 13, 2014

(30) Foreign Application Priority Data
Jun. 15, 2011 (IN) 2027/CHE/2011

(51) Int. Cl. C07D 317/44; C07D 487/04

(52) U.S. Cl. CPC C07D 317/44; C07D 487/04
USPC 544/254; 549/437

(57) ABSTRACT

Disclosed herein is an improved process for the preparation of substituted cyclopentanamine derivatives, which are useful intermediates in the preparation of triazolo[4,5-d]pyrimidine compounds. Particularly described is an improved, commercially viable and industrially advantageous process for the preparation of a ticagrelor intermediate, [3aR-(3aα,4α,6α,6aα]-2-[[6-amino-2,2-dimethyltetrahydro-4H-cyclopenta-1,3-dioxol-4-yl]oxy]-ethanol, alternatively named, 2-[[(3aR,4S,6R,6aS)-6-amino-2,2-dimethyltetrahydro-3aH-cyclopenta[d][1,3]-dioxol-4-yl]oxy]-1-ethanol.

### FIELD OF THE DISCLOSURE

The present disclosure relates to an improved process for the preparation of substituted cyclopentanamine derivatives, which are useful intermediates in the preparation of triazolo[4,5-d]pyrimidine compounds. The present dislosure particularly relates to an improved, commercially viable and industrially advantageous process for the preparation of a ticagrelor intermediate, [3aR-(3aα,4α,6α,6aα)]-2-[[6-amino-2,2-dimethyltetrahydro-4H-cyclopenta-1,3-dioxol-4-yl]oxy]-ethanol, alternatively named, 2-[[(3aR,4S,6R,6aS)-6-amino-2,2-dimethyltetrahydro-3aH-cyclopenta[d][1,3]-dioxol-4-yl]oxy]-1-ethanol.

### BACKGROUND

U.S. Pat. Nos. 6,251,910 and 6,525,060 disclose a variety of triazolo[4,5-d]pyrimidine derivatives, processes for their preparation, pharmaceutical compositions comprising the derivatives, and method of use thereof. These compounds act as P_{2T} (P2Y_{ADP} or P2T_{AC}) receptor antagonists and they are indicated for use in therapy as inhibitors of platelet activation, aggregation and degranulation, promoters of platelet disaggregation and anti-thrombotic agents. Among them, Ticagrelor, [1S-(1α,2α,3β(1S*,2R*),5β)]-3-[7-[2-(3,4-difluorophenyl)cyclopropyl]amino]-5-(propyl thio)-3H-1,2,3-triazolo[4,5-d]pyrimidin-3-yl)-5-(2-hydroxyethoxy)-cyclopentane-1,2-diol, acts as Adenosine uptake inhibitor, Platelet aggregation inhibitor, P2Y12 purinoceptor antagonist and Coagulation inhibitor. It is indicated for the treatment of thrombosis, angina, Ischemic heart diseases and coronary artery diseases. Ticagrelor is represented by the following structural formula I:

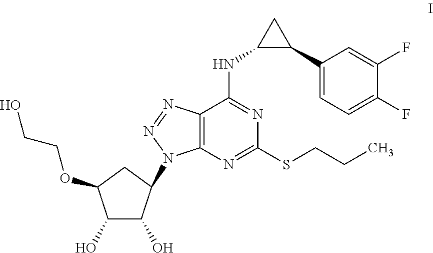 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]

Ticagrelor is the first reversibly binding oral adenosine diphosphate (ADP) receptor antagonist and is chemically distinct from thienopyridine compounds like clopidogrel. It selectively inhibits P2Y12, a key target receptor for ADP. ADP receptor blockade inhibits the action of platelets in the blood, reducing recurrent thrombotic events. The drug has shown a statistically significant primary efficacy against the widely prescribed clopidogrel (Plavix) in the prevention of cardiovascular (CV) events including myocardial infarction (heart attacks), stroke, and cardiovascular death in patients with acute coronary syndrome (ACS).

Various processes for the preparation of pharmaceutically active triazolo[4,5-d]pyrimidine cyclopentane compounds, preferably ticagrelor, their enantiomers, and their pharmaceutically acceptable salts are disclosed in U.S. Pat. Nos. 6,251,910; 6,525,060; 6,974,868; 7,067,663; 7,122,695 and 7,250,419; U.S. Patent application Nos. 2007/0265282, 2008/0132719 and 2008/0214812; European Patent Nos. EP0996621 and EP1135391; and PCT Publication Nos. WO2008/018823 and WO2010/030224.

One of the useful intermediates in the synthesis of pharmaceutically active triazolo[4,5-d]pyrimidine cyclopentane derivatives is the substituted cyclopentanamine derivative of formula II:

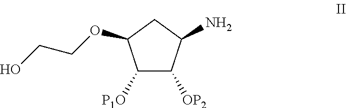

wherein P₁ and P₂ both represents H or a protecting group, or P₁ and P₂ together with the atoms to which they are attached form an alkylidene ring such as a methylidene or isopropylidene ring.

In the preparation of ticagrelor, [3aR-(3aα,4α,6α,6aα)]-2-[[6-amino-2,2-dimethyl tetrahydro-4H-cyclopenta-1,3-dioxol-4-yl]oxy]-ethanol of formula IIa:

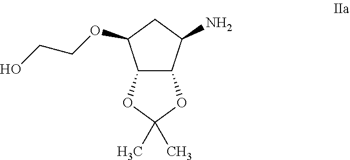

is a key intermediate.

According to the U.S. Pat. No. 6,525,060 (hereinafter referred to as the '060 patent), the substituted cyclopentanamine derivatives of formula II, specifically [3aR-(3aα,4α,6α,6aα)]-2-[[6-amino-2,2-dimethyltetrahydro-4H-cyclopenta-1,3-dioxol-4-yl]oxy]-ethanol of formula IIa, is prepared by a process as depicted in the following scheme 1:

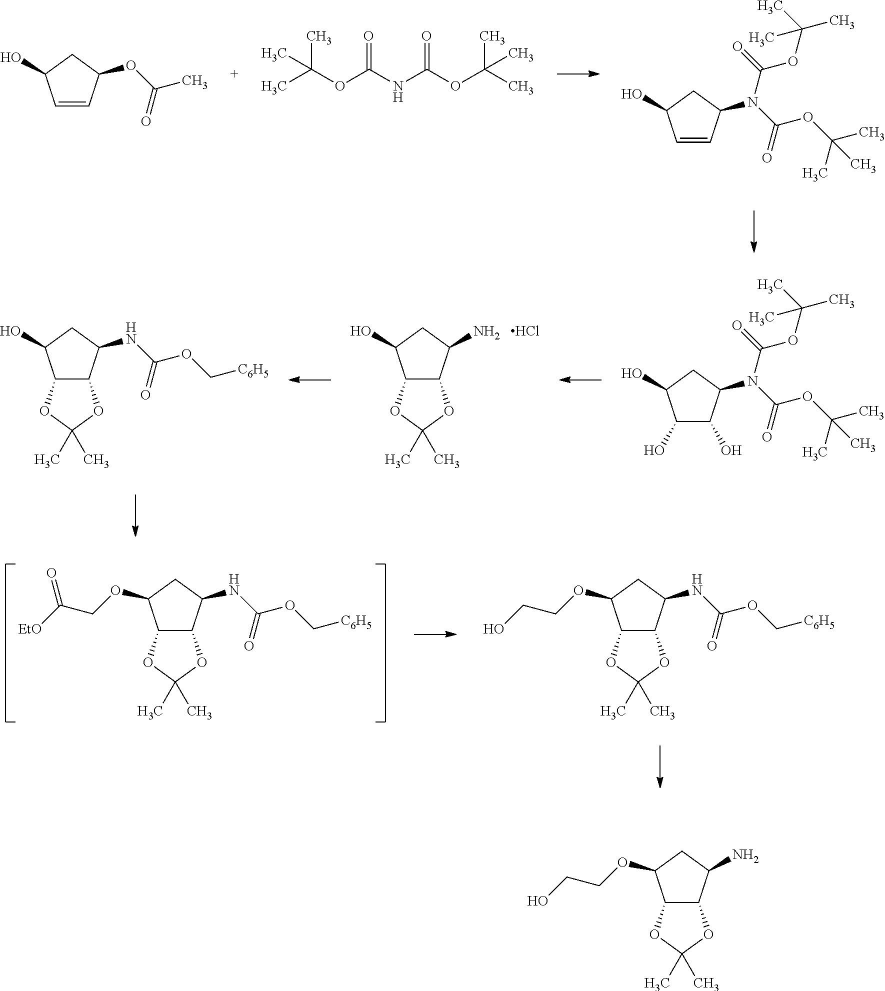

According to the '060 patent, the [3aR-(3aα,4α,6α,6aα)]-2-[[6-amino-2,2-dimethyltetrahydro-4H-cyclopenta-1,3-dioxol-4-yl]oxy]-ethanol is prepared by reacting imidodicarbonic acid bis-(1,1-dimethylethyl)ester with (1S-cis)-4-acetoxy-2-cyclopenten-1-ol in the presence of sodium hydride and tetrakis-(triphenylphosphine)palladium in tetrahydrofuran to produce a reaction mass, followed by column chromatographic purification (SiO₂, ethyl acetate:hexane 1:9 as eluant) to produce (1R-cis)-bis(1,1-dimethylethyl)-4-hydroxy-2-cyclopentenylimidodicarbonate, which is then subjected to oxidation in the presence of osmium tetroxide (2.5% solution in t-butanol) and N-methylmorpholine-N-oxide in a solvent mixture containing tetrahydrofuran and water for 4 days to produce a reaction mass, followed by column chromatographic purification (SiO₂, ethyl acetate:hexane 1:1 as eluant) to produce [1R-(1α,2β,3β,4α)]-2,3,4-trihydroxy-cyclopentenylimidodicarbonic acid, bis(1,1-dimethylethyl)ester. The resulting trihydroxy compound is stirred with hydrochloric acid and methanol for 18 hours to produce a reaction mixture, followed by evaporation to produce a colorless powder, which is then reacted with 2,2-dimethoxypropane and concentrated hydrochloric acid in acetone to produce [3aR-(3aα,4α,6α,6aα)]-6-amino-tetrahydro-2,2-dimethyl-4H-cycloplenta-1,3-dioxol-4-ol hydrochloride salt. The resulting hydroxy compound is then reacted with benzyl chloroformate in the presence of potassium carbonate in 4-methyl-2-pentanone and water to produce a reaction mass, followed by usual work up and subsequent column chromatographic purification (SiO₂, dichloromethane: methanol, 95:5 to 90:10 as eluant) to produce [3aS-(3aα,4α,6α,6aα)]-[tetrahydro-6-hydroxy-2,2-dimethyl-4H-cyclopenta-1,3-dioxol-4-yl]-carbamic acid, phenylmethyl ester. The [3aS-(3aα,4α,6α,6aα)]-[tetrahydro-6-hydroxy-2,2-dimethyl-4H-cyclopenta-1,3-dioxol-4-yl]-carbamic acid phenylmethyl ester is reacted with ethyl bromoacetate in the presence of potassium tert-butoxide in tetrahydrofuran to produce a reaction mass containing as ester intermediate, which is, in-situ, subjected to reduction with Lithium borohydride in the presence of glacial acetic acid, followed by usual work up and subsequent column chromatographic purification (SiO₂, ethyl acetate:hexane, 25:75 to 50:50 as eluant) to produce [3aS-(3aα,4α,6α,6aα)]-[2,2-dimethyl-6-(2-hydroxyethoxy)-tetrahydro-4H-cyclopenta-1,3-dioxol-4-yl]-carbamic acid, phenylmethyl ester. The phenylmethyl ester is then hydrogenated using 5% palladium on charcoal catalyst in ethanol to produce the [3aR-(3aα,4α,6α,6aα)]-2-[[6-amino-2,2-dimethyltetrahydro-4H-cyclopenta-1,3-dioxol-4-yl]oxy]-ethanol.

The process for the preparation of [3aR-(3aα,4α,6α,6aα)]-2-[[6-amino-2,2-dimethyltetrahydro-4H-cyclopenta-1,3-dioxol-4-yl]oxy]-ethanol described in the '060 patent suffers from several disadvantages since the process involves lengthy, tedious and cumbersome procedures such as the use of hazardous and explosive materials like sodium hydride, additional and expensive reagents like tetrakis(triphenylphosphine)palladium, use of multiple and hazardous solvents, longer reaction times (for example the oxidation reaction requires 4 days for completion), use of expensive column chromatographic purifications at various stages, resulting in low selectivity and reactivity, use of expensive raw materials, and thus resulting in low overall yields of the product. Moreover, methods involving column chromatographic purifications are generally undesirable for large-scale operations, thereby making the process commercially unfeasible.

U.S. Pat. No. 7,393,962 (hereinafter referred to as the '962 patent) discloses a process for the alkylation of substituted cyclopentanamine derivatives by reaction of substituted cyclopentanols with an alkyl or arylbromoacetate using a metal alkoxide.

The process described in the '962 patent suffers with poor selectivity thus resulting in a poor product quality intern yield.

Various processes for syntheses of free amine or hydrochloride salt of substituted cyclopentanoloamine derivatives are apparently disclosed in PCT Publication No. WO99/05142; Synthetic communications 31 (2001) 18, 2849-2854; Tetrahedron, 1997, 53, 3347; HeIv. Chim. Acta, 1983, 66, 1915; Tetrahedron, 1997, 53, 3347; and Tetrahedron Lett., 2000, 41, 9537.

U.S. Pat. No. 7,067,663, PCT Publication Nos. WO2009/064249 and WO2010/030224 disclose L-tartrate, dibenzoyl-L-tartrate and oxalate salts of substituted cyclopentanoloamine derivatives.

Based on the aforementioned drawbacks, the prior art processes have been found to be unsuitable for the preparation of substituted cyclopentanamine derivatives of formula II at lab scale and in commercial scale operations.

A need remains for an improved and commercially viable process of preparing substituted cyclopentanamine derivatives of formula II with high yields and purity, to resolve the problems associated with the processes described in the prior art, and that will be suitable for large-scale preparation. Desirable process properties include non-hazardous, environmentally friendly and easy to handle reagents, reduced reaction times, reduced cost, greater simplicity, increased purity, and increased yield of the product, thereby enabling the production of triazolo[4,5-d]pyrimidine compounds, preferably ticagrelor, and their pharmaceutically acceptable acid addition salts in high purity and in high yield.

### SUMMARY

In one aspect, provided herein is a novel, efficient, industrially advantageous and environmentally friendly process for the preparation of substituted cyclopentanamine derivatives using novel intermediates, in high yield and with high chemical and enantiomeric purity. Moreover, the process disclosed herein involves less hazardous and easy to handle reagents, reduced reaction times and reduced synthesis steps. The process disclosed herein avoids the use of tedious and cumbersome procedures described in the prior art and is therefore efficient and convenient to operate on a commercial scale.

In another aspect, provided herein is a novel, efficient, industrially advantageous and environmentally friendly process for the preparation of ticagrelor intermediate, [3aR-(3aα,4α,6α,6aα)]-2-[[6-amino-2,2-dimethyltetrahydro-4H-cyclopenta-1,3-dioxol-4-yl]oxy]-ethanol, in high yield and with high chemical and enantiomeric purity.

In another aspect, the highly pure [3aR-(3aα,4α,6α,6aα)]-2-[[6-amino-2,2-dimethyltetrahydro-4H-cyclopenta-1,3-dioxol-4-yl]oxy]-ethanol obtained by the process disclosed herein has a total purity, which includes both chemical and enantiomeric purity, of greater than about 95%, specifically greater than about 98%, more specifically greater than about 99%, and most specifically greater than about 99.5% as measured by HPLC.

In another aspect, the present disclosure also encompasses the use of pure [3aR-(3aα,4α,6α,6aα)]-2-[[6-amino-2,2-dimethyltetrahydro-4H-cyclopenta-1,3-dioxol-4-yl]oxy]-ethanol obtained by the process disclosed herein for preparing ticagrelor.

The process for the preparation of substituted cyclopentanamine derivatives disclosed herein has the following advantages over the processes described in the prior art:

i) the overall process involves shorter reaction times and reduced process steps;
ii) the process avoids the use of hazardous and explosive chemicals like sodium hydride; iii) the process avoids the use of tedious and cumbersome procedures like column chromatographic purifications and multiple isolations;
iv) the process avoids the use of expensive materials;
v) the process involves easy work-up methods and simple isolation processes and reduction in chemical waste;
vi) high quality product is obtained without additional purifications; and
vii) the overall yield of the product is increased.

### DETAILED DESCRIPTION

According to one aspect, there is provided a process for the preparation of a substituted cyclopentanamine derivative of formula II:

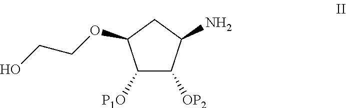

or an acid addition salt thereof; wherein P₁ and P₂ both represents hydrogen or a protecting group, or P₁ and P₂ together with the atoms to which they are attached form an alkylidene ring such as a methylidene or isopropylidene ring; comprising:
a) reacting a cyclopentanol compound of formula III:

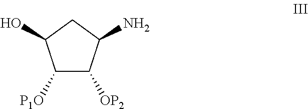

wherein P₁ and P₂ are as defined above, with a substituted benzyl compound of formula IV:

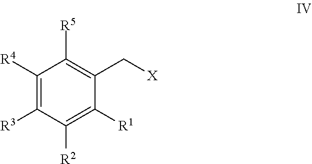

wherein ‘X’ is a leaving group, selected from the group consisting of mesyl, tosyl, Cl, Br and I; and wherein R¹, R², R³, R⁴ and R⁵ are, each independently, selected from hydrogen, F, Cl, Br, I, nitro, C₁-C₃-alkyl, and C₁-C₃-alkoxy substituents; in the presence of a base in a first solvent to produce a benzyl protected compound of formula V:

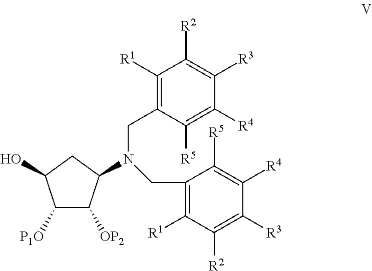

wherein P₁, P₂, R¹, R², R³, R⁴ and R⁵ are as defined above;
b) reacting the compound of formula V with a compound of formula VI:

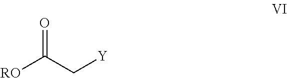

wherein ‘Y’ is a leaving group, selected from the group consisting of mesyl, tosyl, Cl, Br and I; R is C₁₋₆ straight or branched alkyl, or a benzyl group, wherein the phenyl ring of benzyl group is optionally substituted with one or more of the nitro, S(O)₂(C₁₋₄ alkyl), cyano, C₁₋₄ alkyl, C₁₋₄ alkoxy, C(O)(C₁₋₄ alkyl), N(C₁₋₆alkyl)₂, CF₃ or OCF₃;
in the presence of an organic or inorganic base in a second solvent to produce an ester compound of formula VII:

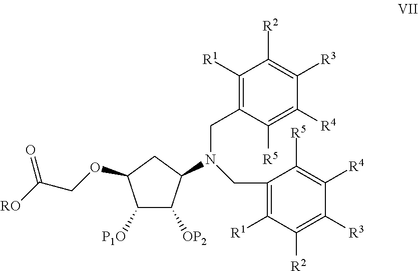

wherein P₁, P₂, R, R¹, R², R³, R⁴ and R⁵ are as defined above;
c) debenzylating the ester compound of formula VII with a debenzylation agent in the presence of third solvent to produce a cyclopentamine ester compound of formula VIII:

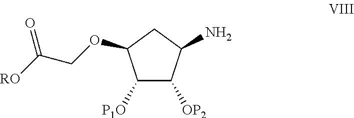

wherein P₁, P₂ and R are as defined above; and optionally converting the compound of formula VIII obtained into an acid addition salt thereof by contacting with a suitable acid; and
d) reducing the compound of formula VIII or an acid addition salt thereof in a fourth solvent to produce the substituted cyclopentanamine derivative of formula II, and optionally converting the compound of formula II obtained into an acid addition salt thereof.

Exemplary protecting groups P₁ and P₂ in the compounds of formulae II, III, V, VII and VIII are C₁₋₆ alkyl (preferably methyl), benzyl, (C₁₋₆ alkyl)₃Si (preferably t-butyldimethylsilyl), and a C(O)C₁₋₆ alkyl group such as acetyl.

In one embodiment, the two groups P₁ and P₂ together with the atoms to which they are attached form an isopropylidene ring.

In another embodiment, the two groups P₁ and P₂ can form an alkoxymethylidene ring such as ethoxymethylidene.

Protecting groups can be added and removed using known reaction conditions. The use of protecting groups is fully described in ‘Protective Groups in Organic Chemistry’, edited by J W F McOmie, Plenum Press (1973), and ‘Protective Groups in Organic Synthesis’, 2^{nd} edition, T W Greene & P G M Wutz, Wiley-Interscience (1991).

In one embodiment, the leaving group ‘X’ in the compound of formula IV is Cl or Br, and more specifically Br.

In another embodiment, the groups R¹, R², R³, R⁴ and R⁵ in the compounds of formulae IV, V and VII are hydrogen.

In another embodiment, the leaving group ‘Y’ in the compound of formula VI is Cl or Br, and more specifically Br. In another embodiment, the group ‘R’ in the compounds of formulae VI, VII and VIII is tert-butyl.

In one embodiment, a most specific substituted cyclopentanamine derivative of formula II prepared by the process described herein is [3aR-(3aα,4α,6α,6aα)]-2-[[6-amino-2,2-dimethyl tetrahydro-4H-cyclopenta-1,3-dioxol-4-yl]oxy]-ethanol of formula IIa (formula II, wherein P₁ and P₂ together with the atoms to which they are attached form an isopropylidene ring):

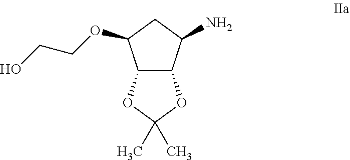

Exemplary bases used in step-(a) include, but are not limited to, sodium hydroxide, sodium bicarbonate, potassium hydroxide, lithium hydroxide, potassium carbonate, sodium carbonate, cesium carbonate, cesium hydroxide, magnesium hydroxide, calcium hydroxide, calcium oxide, triethyl amine, N,N-diisopropylethylamine, N-methylpiperidine, pyridine, N,N-dimethylaminopyridine, N-methylmorpholine and azabicyclononane. Specifically, the base is selected from the group consisting of sodium hydroxide, sodium bicarbonate, potassium hydroxide, lithium hydroxide, potassium carbonate and sodium carbonate; and more specifically potassium carbonate and sodium carbonate.

In one embodiment, the reactions can be homogenous or heterogeneous.

Exemplary first solvents used in step-(a) include, but are not limited to, water, a protic solvent, a solvent miscible with water, a dipolar a protic solvent, and mixtures thereof. The term solvent also includes mixtures of solvents.

Specifically, the first solvent is selected from the group consisting of water, methanol, ethanol, isopropyl alcohol, tetrahydrofuran, acetonitrile, dimethylformamide, dimethylacetamide, tetramethyl urea and its cyclic analog, dimethylsulfoxide, N-methylpyrrolidone, sulfolane, nitromethane, and mixtures thereof; and most specifically a mixture of water and ethanol.

Specific alkylating agents used in step-(a) are benzyl bromide or benzyl chloride or monosubstituted aralkyl halides or polysubstituted aralkyl halides. Sulfate or sulfonate esters are also suitable reagents to provide the corresponding benzyl analogs and they can be preformed from the corresponding benzyl alcohol or formed in situ by methods well known to those skilled in the art. Trityl, benzhydryl, substituted trityl, substituted benzhydryl, allyl and substituted allyl groups, independently, are also effective amine protecting groups. Their halide derivatives can also be prepared from the corresponding alcohols by methods well known to those skilled in the art such as treatment with thionyl chloride or bromide or with phosphorus tri- or pentachloride, bromide or iodide or the corresponding phosphoryl trihalide. Examples of groups that can be substituted on the aryl ring include alkyl, alkoxy, hydroxy, nitro, halo and alkylene, amino, mono- and dialkyl amino and acyl amino, acyl and water solubilizing groups such as phosphonium salts and ammonium salts. The aryl ring can be derived from, for example, benzene, napthelene, indane, anthracene, 9-phenyl-9H-fluorene, durene, phenanthrene and the like.

In one embodiment, the alkylation reaction in step-(a) is carried out at a temperature of about 0° C. to about 100° C., specifically at a temperature of about 20° C. to about 80° C., and more specifically at a temperature of about 35° C. to about 70° C. The reaction time may vary between about 2 hour to about 12 hours, specifically about 3 hours to about 10 hours, and more specifically about 6 hours to about 9 hours. The reaction may be carried out under an inert atmosphere such as nitrogen or argon, or normal or dry air, under atmospheric pressure or in a sealed reaction vessel under positive pressure.

Alternatively, the compound of Formula V can also be prepared by reductive alkylation by, for example, compounds and intermediates formed from the addition of an aldehyde with the amine and a reducing agent; reduction of a Schiff base, carbinolamine or enamine; or reduction of an acylated amine derivative. Reducing agents include metals (platinum, palladium, palladium hydroxide, palladium on carbon, platinum oxide, rhodium and the like) with hydrogen gas or hydrogen transfer molecules such as cyclohexene or cyclohexadiene; or hydride agents such as lithium aluminumhydride, sodium borohydride, lithium borohydride, sodium cyanoborohydride, diisobutylaluminum hydride or lithium tri-tert-butoxyaluminum hydride.

Additives such as sodium or potassium bromide, sodium or potassium iodide can catalyze or accelerate the rate of amine alkylation, especially when benzyl chloride is used as the nitrogen alkylating agent.

In one embodiment, the reaction in step-(a) is optionally carried out via phase transfer catalysis wherein the amine to be protected and the nitrogen alkylating agent are reacted with a base in a solvent mixture in the presence of a phase transfer reagent, catalyst or promoter. The solvent mixture can consist of, for example, toluene, benzene, ethylene dichloride, cyclohexane, methylene chloride or the like with water, or an aqueous solution of an organic water miscible solvent such as tetrahydrofuran. Examples of phase transfer catalysts include tetrabutylammonium chloride, tetrabutylammonium iodide, tetrabutylammonium bromide, tetrabutylammonium hydroxide, tri-butyloctylammonium chloride, dodecyltrihexylammonium hydroxide, methyltrihexylammonium chloride, and the like.

The reaction mass containing the alkylated compound of formula V obtained in step-(a) may be subjected to usual work up such as a washing, an extraction, a pH adjustment, an evaporation or a combination thereof. The reaction mass may be used directly in the next step to produce the compound of formula VII, or the alkylated compound of formula V may be isolated and then used in the next step.

In one embodiment, the alkylated compound of formula V is isolated from a suitable solvent by conventional methods such as cooling, seeding, partial removal of the solvent from the solution, by adding an anti-solvent to the solution, evaporation, vacuum distillation, or a combination thereof.

The solvent used to isolate the alkylated compound of formula V is selected from the group consisting of water, tetrahydrofuran, 2-methyl tetrahydrofuran, diisopropyl ether, methyl tert-butyl ether, n-pentane, n-hexane, n-heptane, cyclohexane, toluene, xylene, dichloromethane, dichloroethane, chloroform, and mixtures thereof; and most specifically, toluene, n-heptane, dichloromethane, 2-methyl tetrahydrofuran and mixtures thereof.

In another embodiment, the reaction mass containing the alkylated compound of formula V obtained is concentrated and then taken for next step.

Exemplary bases used in step-(b) include, but are not limited to, a metal hydroxide, a metal hydride, a metal amide, a metal alkoxide, an alkyl lithium, a metal diisopropylamide, and a metal methylsilazide.

In one embodiment, the base used in step-(b) is selected from the group consisting of sodium hydroxide, potassium hydroxide, lithium hydroxide, cesium hydroxide, magnesium hydroxide, calcium hydroxide, sodium hydride, lithium hydride, potassium hydride, sodamide, lithium amide, potassium amide, sodium methoxide, potassium tert-butoxide, sodium tert-butoxide, sodium tert-pentoxide, lithium tert-butoxide, n-butyl lithium, n-hexyl lithium, lithium diisopropylamide, sodium diisopropyl amide, potassium diisopropyl amide, lithium hexamethyldisilazide, sodium hexamethyldisilazide and potassium hexamethyldisilazide.

In one embodiment, the second solvent used in step-(b) is selected from the group consisting of acetone, methylethyl ketone, methylisobutyl ketone, methyltert-butyl ketone, acetonitrile, tetrahydrofuran, 2-methyl tetrahydrofuran, 1,4-dioxane, diethyl ether, diisopropyl ether, methyltert-butyl ether, monoglyme, diglyme, n-pentane, n-hexane, n-heptane, cyclohexane, toluene, xylene, N,N-dimethylformamide, N,N-dimethylacetamide, dimethylsulfoxide, N-methylpyrrolidone, and mixtures thereof.

Additives such as sodium bromide, potassium bromide, sodium iodide and potassium iodide can catalyze or accelerate the rate of alkylation reaction, especially when Cl is used as a leaving group in the alkylating agent of formula VI.

In one embodiment, the reaction in step-(b) is optionally carried out via phase transfer catalysis wherein the alcohol compound and the alkylating agent are reacted with a base in a solvent mixture in the presence of a phase transfer reagent, catalyst or promoter. The solvent mixture can consist of, for example, toluene, benzene, ethylene dichloride, cyclohexane, methylene chloride and the like with water or an aqueous solution of an organic water miscible solvent such as tetrahydrofuran. The phase transfer catalysts are selected from the group as described above.

In one embodiment, the alkylation reaction in step-(b) is carried out at a temperature of about −50° C. to about 90° C., specifically at a temperature of about −20° C. to about 50° C., and more specifically at a temperature of about 0° C. to about 10° C. The reaction time may vary between about 30 minutes to about 6 hours, specifically about 1 hour to about 5 hours, and more specifically about 2 hours to about 4 hours.

The reaction mass containing the alkylated product obtained in step-(b) may be subjected to usual work up methods as described above. The reaction mass may be used directly in the next step, or the compound of formula VII may be isolated, or optionally purified, and then used in the next step.

In one embodiment, the compound of formula VII is isolated and/or purified from a suitable solvent by conventional methods as described above.

In one embodiment, the third solvent used in step-(c) include, but are not limited to, methanol, ethanol, isopropyl alcohol, n-propanol, n-butanol, tetrahydrofuran, 2-methyl tetrahydrofuran, 1,4-dioxane, diethyl ether, diisopropyl ether, methyl tert-butyl ether, dimethoxyethane, diethoxyethane, toluene, xylene, dichloromethane, dichloroethane, chloroform, and mixtures thereof; and most specifically methanol, ethanol, 2-methyl tetrahydrofuran, tetrahydrofuran, and mixtures thereof.

In one embodiment, the deprotection in step-(c) comprises the single-step removal of the benzyl protecting group. The deprotection is carried out either by catalytic hydrogenation under high pressure (about 40 to about 100 psi), specifically at a temperature of about 40 to about 80° C., and more specifically in the presence of acetic acid; or by catalytic transfer hydrogenation (CTH) and specifically in acetic acid. Exemplary hydrogenation catalysts include, but are not limited to, Pd/C, Pd(OH)₂ and the like.

In another embodiment, the benzyl group can be removed by catalytic hydrogen transfer process. Specifically, the catalytic transfer hydrogenation reagents are selected from the group consisting of 1,4-cyclohexadiene, cyclohexene, ammonium formate, formic acid, sodium formate, hydrazine, 1,3-cyclohexadiene and trialkylammonium formates, and combinations comprising the foregoing reagents.

In one embodiment, the reaction in step-(c) is carried out at a temperature of about −5° C. to about 80° C. for at least 30 minutes, specifically at a temperature of about 10° C. to about 70° C. for about 2 hours to about 16 hours, and most specifically at about 30° C. to about 60° C. for about 8 hours to about 15 hours.

The reaction mass containing the substituted cyclopentanamine ester derivative of formula VIII or a stereochemically isomeric form or a mixture of stereochemically isomeric forms thereof obtained in step-(c) may be subjected to usual work up, followed by isolating and/or recovering from a suitable solvent by the methods as described above, wherein the solvent is selected from the group consisting of water, an alcohol, a ketone, an ester, an aliphatic ether, a hydrocarbon solvent, a chlorinated hydrocarbon, and mixtures thereof. Specifically, the solvent is selected from the group consisting of water, methanol, ethanol, acetone, isopropanol, ethyl acetate, butyl acetate, dichloromethane, diethyl ether, diisopropyl ether, methyl tert-butyl ether, toluene, n-heptane, n-pentane, n-hexane, cyclohexane, and mixtures thereof.

The reaction mass containing the substituted cyclopentanoloamine ester derivatives of formula VIII or a stereochemically isomeric form or a mixture of stereochemically isomeric forms thereof obtained in step-(c) may be subjected to usual work up methods as described above. The reaction mass may be used directly in the next step, or the compound of formula VIII may be isolated, or optionally purified or converted into its acid addition salt thereof, and then used in the next step.

In one embodiment, the compound of formula VIII is isolated and/or purified from a suitable solvent by the conventional methods as described above.

In a preferred embodiment, the reaction mass containing the substituted cyclopentanoloamine ester derivatives of formula VIII or a stereochemically isomeric form or a mixture of stereochemically isomeric forms thereof obtained in step-(c) may be subjected to usual work up as described above and then converted into its acid addition salt by reacting with a suitable acid in a suitable solvent, wherein the solvent is selected from the group consisting of water, an alcohol, a ketone, an ether, a nitrile solvent, a polar aprotic solvent, and mixtures thereof. Specific solvents are alcohols and more specifically isopropanol.

The acid used for preparing the acid addition salts of the compound of formula VIII is selected from the group consisting of hydrochloric acid, hydrobromic acid, sulfuric acid, nitric acid, phosphoric acid, acetic acid, propionic acid, oxalic acid, succinic acid, maleic acid, fumaric acid, methanesulfonic acid, benzenesulfonic acid, toluenesulfonic acid, citric acid, glutaric acid, citraconic acid, glutaconic acid, L-(+)-tartaric acid, D-(−)-tartaric acid, dibenzoyl-L-tartaric acid, di-p-toluoyl-L-tartaric acid, di-p-anisoyl-L-tartaric acid, (R)-(−)-α-methoxyphenyl acetic acid, L-malic acid, malonic acid, mandelic acid, (1S)-(+)-10-camphorsulfonic acid. The salt derived from L-(+)-tartaric acid is particularly preferred.

Exemplary reducing agents used in step-(d) include, but are not limited to, lithium aluminumhydride, lithium borohydride, sodium borohydride, borane, lithium tri-ter-butoxyaluminum hydride, borane-THF complex, diisobutylaluminum hydride (DIBAL-H), sodium bis(2-methoxyethoxy)aluminum hydride (Vitride®). Specifically, the reducing agent is selected from the group consisting of lithium borohydride, diisobutylaluminum hydride (DIBAL-H) and sodium bis(2-methoxyethoxy)aluminum hydride (Vitride®) in toluene.

Exemplary fourth solvents used in step-(d) include, a hydrocarbon, a cyclic ether, an aliphatic ether, a chlorinated hydrocarbon and the like, and mixtures thereof.

In one embodiment, the fourth solvent is selected from the group consisting of tetrahydrofuran, 2-methyl tetrahydrofuran, 1,4-dioxane, diethyl ether, diisopropyl ether, methyl tert-butyl ether, n-pentane, n-hexane, n-heptane, cyclohexane, toluene, xylene, dichloromethane, dichloroethane, chloroform, and mixtures thereof; and most specifically, toluene, dichloromethane, 2-methyl tetrahydrofuran, tetrahydrofuran, and mixtures thereof.

In one embodiment, the reaction in step-(d) is carried out at a temperature of about −20° C. to about 80° C., specifically at a temperature of about −10° C. to about 60° C., and most specifically at about 0° C. to about 35° C. In another embodiment, the reaction is carried out for about 1 hour to about 30 hours, specifically for about 5 hours to about 26 hours, and most specifically for about 15 hours to about 25 hours.

The reaction mass containing the substituted cyclopentanamine derivative of formula II or a stereochemically isomeric form or a mixture of stereochemically isomeric forms thereof obtained in step-(d) may be subjected to usual work up, and followed by isolating and/or recovering from a suitable solvent by the methods as described above, wherein the solvent is selected from the group consisting of water, an alcohol, a ketone, an ester, an aliphatic ether, a hydrocarbon solvent, a chlorinated hydrocarbon, and mixtures thereof. Specifically, the solvent is selected from the group consisting of water, methanol, ethanol, acetone, isopropanol, ethyl acetate, butyl acetate, dichloromethane, diethyl ether, diisopropyl ether, methyl tert-butyl ether, toluene, n-heptane, n-pentane, n-hexane, cyclohexane, and mixtures thereof.

The use of inexpensive, non-explosive, non-hazardous, readily available and easy to handle reagents and solvents allows the process disclosed herein to be suitable for preparation of the substituted cyclopentanamine derivatives of formula II or a stereochemically isomeric form or a mixture of stereochemically isomeric forms thereof at lab scale and in commercial scale operations.

Acid addition salts of the compounds of formula II can be prepared in high purity by using the substantially pure substituted cyclopentanamine derivatives of formula II or a stereochemically isomeric form or a mixture of stereochemically isomeric forms thereof obtained by the method disclosed herein, by known methods.

The acid addition salts of substituted cyclopentanamine derivatives of formula II or a stereochemically isomeric form or a mixture of stereochemically isomeric forms thereof are derived from a therapeutically acceptable acid selected from the group consisting of hydrochloric acid, hydrobromic acid, sulfuric acid, nitric acid, phosphoric acid, acetic acid, propionic acid, oxalic acid, succinic acid, maleic acid, fumaric acid, methanesulfonic acid, benzenesulfonic acid, toluenesulfonic acid, citric acid, glutaric acid, citraconic acid, glutaconic acid, tartaric acid, dibenzoyl-L-tartaric acid, di-p-toluoyl-L-tartaric acid, di-p-anisoyl-L-tartaric acid, (R)-(−)-α-methoxyphenyl acetic acid, L-malic acid, malonic acid, mandelic acid, (1S)-(+)-10-camphorsulfonic acid.

The term “substantially pure substituted cyclopentanoloamine derivatives” refers to the substituted cyclopentanoloamine derivatives having a total purity, including both stereochemical and chemical purity, of greater than about 95%, specifically greater than about 98%, more specifically greater than about 99%, and still more specifically greater than about 99.5%. The purity is preferably measured by High Performance Liquid Chromatography (HPLC). For example, the purity of the substituted cyclopentanoloamine derivatives obtained by the process disclosed herein is about 95% to about 99%, or about 98% to about 99.5%, as measured by HPLC.

Aptly the process of the present invention is adapted for the preparation of triazolo[4,5-d]pyrimidinecyclopentane compounds, preferably Ticagrelor, and their pharmaceutically acceptable acid addition salts, in high enantiomeric and chemical purity.

Ticagrelor and pharmaceutically acceptable acid addition salts thereof can be prepared in high purity by using the substantially pure [3aR-(3aα,4α,6α,6aα)]-2-[[6-amino-2,2-dimethyl tetrahydro-4H-cyclopenta-1,3-dioxol-4-yl]oxy]-ethanol of formula IIa obtained by the methods disclosed herein, by known methods.

The compounds of formulae V, VII and acid addition salts of VIII are novel and constitute another aspect of the invention.

The use of the intermediate compounds of formulae V, VII and acid addition salts of VIII and their stereochemical isomers and acid addition salts thereof, in the preparation of substituted cyclopentanamine derivatives of formula II or a stereochemically isomeric form or a mixture of stereochemically isomeric forms thereof is novel and forms further aspect of the present invention.

The following examples are given for the purpose of illustrating the present disclosure and should not be considered as limitation on the scope or spirit of the disclosure.

### EXAMPLES

### Example 1

### Preparation of (3aR,4S,6R,6aS)-6-(N,N-Dibenzylamino)-2,2-dimethyltetrahydro-3aH-cyclopenta[d][1,3]dioxol-4-ol

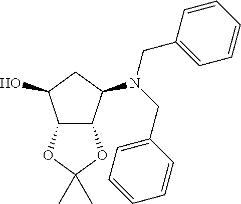 [SMILES: CC1(C)O[C@@H]2[C@H](O1)[C@@H](O)C[C@H]2N(Cc1ccccc1)Cc1ccccc1]

(3aR,4S,6R,6aS)-6-Amino-2,2-dimethyltetrahydro-3aH-cyclopenta[d][1,3]dioxol-4-ol (115 g) was added to a solution of sodium carbonate (246.29 g) in water (450 ml). A solution of benzyl bromide (227.19 g) in denatured ethanol (230 ml) was added to the resulting suspension while maintaining the temperature at about 25-30° C. The resulting mixture was heated at 38-42° C., followed by stiffing for 8 hours at 38-42° C. After completion of the reaction, 25% aqueous ammonia solution (75 ml) and water (1150 ml) were added to the reaction mass, followed by stirring for 15 minutes at 25-30° C. The resulting basic solution was extracted with dichloromethane (2×575 ml), followed by washing the combined dichloromethane layer with water (2×288 ml). The dichloromethane layer was concentrated under reduced pressure while maintaining the temperature at below 40° C. n-Heptane (1380 ml) was added to the concentrated mass, followed by heating at 60-65° C. The resulting solution was cooled to 30-35° C., followed by the addition of seeding (1.15 g). The resulting thick slurry was stirred for 3 hours at 25-30° C., followed by cooling to 0-5° C. The cooled slurry was stirred for 2 hours, followed by isolation of product by filtration. The wet cake was washed with chilled n-heptane (58 ml and 115 ml). The wet cake was further suction dried, followed by drying at 30-35° C. under reduced pressure to obtain 197 g of (3aR,4S,6R,6aS)-6-(N,N-dibenzylamino)-2,2-dimethyltetrahydro-3aH-cyclopenta[d][1,3]dioxol-4-ol as off white solid (Yield: 171.30% w/w; Purity by HPLC: 98.65% by area).

### Example 2

### Preparation of tert-Butyl [[(3aR,4S,6R,6aS)-6-(N,N-Dibenzylamino)-2,2-dimethyl tetrahydro-3aH-cyclopenta[d][1,3]dioxol-4-yl]oxy]acetate

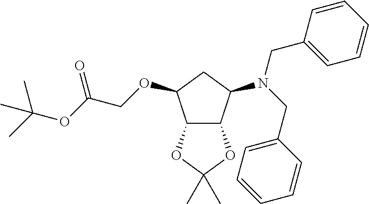 [SMILES: CC(C)(C)OC(=O)CO[C@H]1C[C@@H](N(Cc2ccccc2)Cc2ccccc2)[C@@H]2OC(C)(C)O[C@@H]21]

(3aR,4S,6R,6aS)-6-(N,N-Dibenzylamino)-2,2-dimethyltetrahydro-3aH-cyclopenta [d][1,3]dioxol-4-ol (190 g), dichloromethane (1425 ml) and tert-butyl bromoacetate (227.77 g) were taken into a clean and dry reaction assembly, followed by rinsing of the tert-butyl bromoacetate container with dichloromethane (48 ml). The resulting mass was cooled to 0 to 5° C., followed by the addition of a solution of potassium tert-butoxide in tetrahydrofuran (947 ml, 1M) over a period of 5 hours while maintaining the temperature at about 0 to 5° C. The resulting solution was stirred for 30 minutes at 0 to 5° C. After completion of the reaction, aqueous solution of ammonium chloride (prepared by mixing 190 g of ammonium chloride with 950 ml of water) was added to the reaction mass, followed by stiffing for 15 minutes. The layers were separated and the aqueous layer was extracted with dichloromethane (570 ml), followed by drying of organic layer over sodium sulfate (95 g) and filtering through celite bed. The celite bed was washed with dichloromethane (2×190 ml) and combined with the main filtrate. The filtrate was concentrated under reduced pressure while maintaining the temperature at below 40° C. to obtain 327 g of tert-Butyl [[(3aR,4S,6R,6aS)-6-(N,N-Dibenzylamino)-2,2-dimethyltetrahydro-3 aH-cyclopenta[d][1,3] dioxol-4-yl]oxy]acetate, which was directly used in the next step (Yield: 172.1% w/w; Purity by HPLC: 94.03% by area).

### Example 3

### Preparation of tert-butyl [[(3aR,4S,6R,6aS)-6-Amino-2,2-dimethyltetrahydro-3aH-cyclopenta[d][1,3]dioxol-4-yl]oxy]acetate L-(+)-tartaric acid salt (1:1)

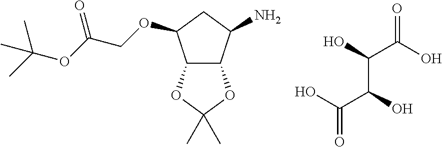 [SMILES: CC(C)(C)OC(=O)CO[C@H]1C[C@@H](N)[C@@H]2OC(C)(C)O[C@@H]21 ; O=C(O)[C@H](O)[C@@H](O)C(=O)O]

A mixture of tert-Butyl [[(3aR,4S,6R,6aS)-6-(N,N-Dibenzylamino)-2,2-dimethyl tetrahydro-3aH-cyclopenta[d][1,3]dioxol-4-yl]oxy]acetate (300 g), palladium on carbon (10% Pd on carbon, 50% wet, 36 g) and denatured ethanol (1750 ml) was taken into an autoclave, followed by nitrogen flushing. The mixture was hydrogenated under hydrogen pressure (70 psi) for 14 hours at 43 to 48° C. After completion of the reaction, the reaction mixture was filtered through celite and the celite bed was washed with denatured ethanol (2×175 ml). The filtrate was concentrated under reduced pressure while maintaining the temperature at about 50 to 55° C., followed by the addition of isopropyl alcohol (437.5 ml) to obtain a clear solution. A solution of L-(+)-tartaric acid (80.5 g) dissolved in isopropyl alcohol (1137.5 ml) was added to the resulting solution over a period of 10 to 15 minutes while maintaining the temperature at about 25 to 30° C., followed by flushing of the container with isopropyl alcohol (87.5 ml). The resulting mass was heated at 50 to 55° C. to obtain a clear solution, followed by gradual cooling to 35 to 40° C. The precipitated mass was stirred for 2 hours at 35 to 40° C., followed by cooling the mass to 20 to 25° C. The cooled slurry was stirred for 10 to 12 hours while maintaining the temperature at about 20 to 25° C., followed by cooling the mass to −5 to 0° C. The cooled slurry was stirred for 2 hours while maintaining the temperature at about −5 to 0° C., followed by the isolation of the product by filtration. The wet cake was washed with chilled isopropyl alcohol (87.5 ml and 175 ml), followed by suction drying. The resulting wet cake was dried under reduced pressure while maintaining the temperature at about 40 to 45° C. to obtain 175 g of tert-butyl [[(3aR,4S,6R,6aS)-6-amino-2,2-dimethyltetrahydro-3aH-cyclopenta[d][1,3]dioxol-4-yl]oxy]acetate L-(+)-tartaric acid salt (1:1) as off white solid.

### Example 4

### Preparation of 2-[[(3aR,4S,6R,6aS)-6-Amino-2,2-dimethyltetrahydro-3aH-cyclopenta[d][1,3]-dioxol-4-yl]oxy]-1-ethanol

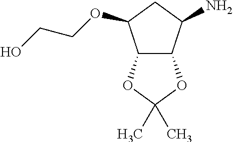

tert-Butyl [[(3aR,4S,6R,6aS)-6-amino-2,2-dimethyltetrahydro-3aH-cyclopenta [d][1,3] dioxol-4-yl]oxy]acetate L-(+)-tartaric acid salt (1:1) (15 g) was suspended in a mixture of dichloromethane (75 ml) and water (45 ml), followed by adjusting the pH to 10 to 10.5 by the addition of aqueous potassium carbonate solution (prepared by dissolving 15 g of potassium carbonate in 30 ml of water) while maintaining the temperature at about 20 to 25° C. The layers were separated and the aqueous layer was extracted with dichloromethane (75 ml). The dichloromethane layers were combined and washed with water (75 ml). The dichloromethane layer was concentrated under reduced pressure while maintaining the temperature at about 40° C. to obtain 8.9 g of free base of tert-butyl [[(3aR,4S,6R,6aS)-6-amino-2,2-dimethyltetrahydro-3aH-cyclopenta[d][1,3]dioxol-4-yl]oxy]acetate.

tert-Butyl [[(3aR,4S,6R,6aS)-6-amino-2,2-dimethyltetrahydro-3aH-cyclopenta [d][1,3]dioxol-4-yl]oxy]acetate free base (1 g, obtained above) was dissolved in dichloromethane (10 ml), followed by the addition of lithium borohydride (0.17 g) under nitrogen atmosphere. The resulting mixture was stirred for 24 hours at 20 to 25° C. After completion of the reaction, acetic acid (1 ml) was added to the reaction mass, followed by stiffing for 15 minutes. To the resulting solution was added solid potassium carbonate (1 g), followed by stirring for 30 minutes. The suspension was filtered and the solid cake was washed with dichloromethane (10 ml), followed by the evaporation of combined dichloromethane filtrate and washing under reduced pressure while maintaining the temperature at about 40° C. to obtain yellow oil which was further purified by column chromatography (silica gel 60-120 mesh, 10% methanol in dichloromethane) to obtain 0.7 g of pure 2-[[(3aR,4S,6R,6aS)-6-Amino-2,2-dimethyltetrahydro-3aH-cyclopenta[d][1,3]-dioxol-4-yl]oxy]-1-ethanol.

1. A process for the preparation of a substituted cyclopentanamine derivative of formula II: 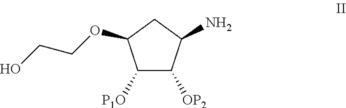
or an acid addition salt thereof; wherein P₁ and P₂ both represent hydrogen or a protecting group, or P₁ and P₂ together with the atoms to which they are attached form an alkylidene ring; comprising:
(a) reacting a cyclopentanol compound of formula III: 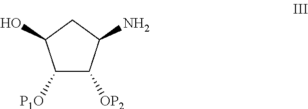
wherein P₁ and P₂ are as defined above, with a substituted benzyl compound of formula IV: 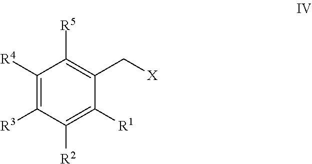
wherein ‘X’ is a leaving group, selected from the group consisting of mesyl, tosyl, Cl, Br and I; and wherein R¹, R², R³, R⁴ and R⁵ are, each independently, selected from hydrogen, F, Cl, Br, I, nitro, C₁-C₃-alkyl, and C₁-C₃-alkoxy substituents;
in the presence of a base in a first solvent to produce a benzyl protected compound of formula V: 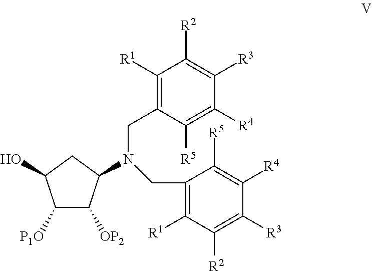
wherein P₁, P₂, R¹, R², R³, R⁴ and R⁵ are as defined above;
(b) reacting the compound of formula V with a compound of formula VI: 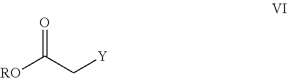
wherein ‘Y’ is a leaving group, selected from the group consisting of mesyl, tosyl, Cl, Br and I; R is C₁₋₆ straight or branched alkyl, or a benzyl group, wherein the phenyl ring of benzyl group is optionally substituted with one or more of the nitro, S(O)₂(C₁₋₄ alkyl), cyano, C₁₋₄ alkyl, C₁₋₄ alkoxy, C(O)(C₁₋₄ alkyl), N(C₁₋₆ alkyl)₂, CF₃ and OCF₃;
in the presence of an organic or inorganic base in a second solvent to produce an ester compound of formula VII: 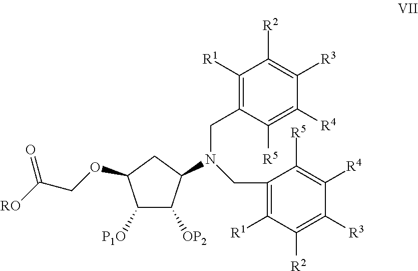
wherein P₁, P₂, R, R¹, R², R³, R⁴ and R⁵ are as defined above;
(c) debenzylating the ester compound of formula VII with a debenzylation agent in the presence of third solvent to produce a cyclopentamine ester compound of formula VIII: 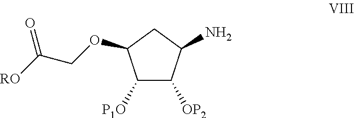
wherein P₁, P₂ and R are as defined above; and optionally converting the compound of formula VIII obtained into an acid addition salt thereof by contacting with a suitable acid; and
(d) reducing the compound of formula VIII or an acid addition salt thereof in a fourth solvent to produce the substituted cyclopentanamine derivative of formula II, and optionally converting the compound of formula II obtained into an acid addition salt thereof.

2. The process of claim 1, wherein the protecting groups P₁ and P₂ in the compounds of formulae II, III, V, VII and VIII are selected from the group consisting of (i) C₁₋₆ alkyl, (ii) benzyl, (iii) (C₁₋₆ alkyl)₃Si, and (iv) C(O)C₁₋₆ alkyl group.

3. The process of claim 1, wherein (i) the two groups P₁ and P₂ together with the atoms to which they are attached form an isopropylidene ring, or (ii) wherein the two groups P₁ and P₂ form an alkoxymethylidene ring.

4. The process of claim 1, wherein the leaving group ‘X’ in the compound of formula IV is Cl or Br.

5. The process of claim 1, wherein the groups R¹, R², R³, R⁴ and R⁵ in the compounds of formulae IV, V and VII are hydrogen.

6. The process of claim 1, wherein the leaving group ‘Y’ in the compound of formula VI is Cl or Br.

7. The process of claim 1, wherein the group ‘R’ in the compounds of formulae VI, VII and VIII is tert-butyl.

8. The process of claim 1, wherein the substituted cyclopentanamine derivative of formula II is [3aR-(3aα,4α,6α,6aα)]-2-[[6-amino-2,2-dimethyl tetrahydro-4H-cyclopenta-1,3-dioxol-4-yl]oxy]-ethanol of formula IIa

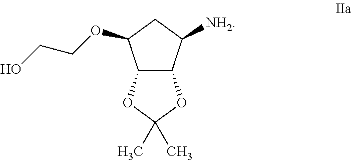

9. The process of claim 1, wherein the base used in step-(a) is selected from the group consisting of sodium hydroxide, sodium bicarbonate, potassium hydroxide, lithium hydroxide, potassium carbonate and sodium carbonate.

10. The process of claim 1, wherein the first solvent is a mixture of water and ethanol.

11. The process of claim 1, wherein the reaction in step-(a) is carried out via phase transfer catalysis, wherein the amine to be protected and the nitrogen alkylating agent are reacted with a base in a solvent mixture in the presence of a phase transfer reagent, catalyst or promoter.

12. The process of claim 1, wherein the solvent used to isolate the alkylated compound of formula V is selected from the group consisting of water, tetrahydrofuran, 2-methyl tetrahydrofuran, diisopropyl ether, methyl tert-butyl ether, n-pentane, n-hexane, n-heptane, cyclohexane, toluene, xylene, dichloromethane, dichloroethane, chloroform, and mixtures thereof.

13. The process of claim 1, wherein the reaction mass containing the alkylated compound of formula V obtained is concentrated and then taken for next step.

14. The process of claim 1, wherein the base used in step-(b) is selected from the group consisting of sodium hydroxide, potassium hydroxide, lithium hydroxide, cesium hydroxide, magnesium hydroxide, calcium hydroxide, sodium hydride, lithium hydride, potassium hydride, sodamide, lithium amide, potassium amide, sodium methoxide, potassium tert-butoxide, sodium tert-butoxide, sodium tert-pentoxide, lithium tert-butoxide, n-butyl lithium, n-hexyl lithium, lithium diisopropylamide, sodium diisopropyl amide, potassium diisopropyl amide, lithium hexamethyldisilazide, sodium hexamethyldisilazide and potassium hexamethyldisilazide.

15. The process of claim 1, wherein the second solvent used in step-(b) is selected from the group consisting of acetone, methylethyl ketone, methylisobutyl ketone, methyltert-butyl ketone, acetonitrile, tetrahydrofuran, 2-methyl tetrahydrofuran, 1,4-dioxane, diethyl ether, diisopropyl ether, methyltert-butyl ether, monoglyme, diglyme, n-pentane, n-hexane, n-heptane, cyclohexane, toluene, xylene, N,N-dimethylformamide, N,N-dimethylacetamide, dimethylsulfoxide, N-methylpyrrolidone, and mixtures thereof.

16. The process of claim 1, wherein the reaction in step-(b) is carried out via phase transfer catalysis wherein the alcohol compound and the alkylating agent are reacted with a base in a solvent mixture in the presence of a phase transfer reagent, catalyst or promoter.

17. The process of claim 1, wherein the third solvent used in step-(c) is selected from the group consisting of methanol, ethanol, isopropyl alcohol, n-propanol, n-butanol, tetrahydrofuran, 2-methyl tetrahydrofuran, 1,4-dioxane, diethyl ether, diisopropyl ether, methyl tert-butyl ether, dimethoxyethane, diethoxyethane, toluene, xylene, dichloromethane, dichloroethane, chloroform, and mixtures thereof.

18. The process of claim 1, wherein the deprotection in step-(c) comprises the single-step removal of the benzyl protecting group, wherein the deprotection is carried out (i) by catalytic hydrogenation under high pressure, or (ii) by catalytic transfer hydrogenation.

19. The process of claim 1, wherein the catalytic transfer hydrogenation reagents are selected from the group consisting of 1,4-cyclohexadiene, cyclohexene, ammonium formate, formic acid, sodium formate, hydrazine, 1,3-cyclohexadiene and trialkylammonium formates, and combinations comprising the foregoing reagents.

20. The process of claim 1, wherein the reaction mass containing the substituted cyclopentanoloamine ester derivatives of formula VIII or a stereochemically isomeric form or a mixture of stereochemically isomeric forms thereof obtained in step-(c) is converted into its acid addition salt by reacting with a suitable acid in a suitable solvent.

21. The process of claim 20, wherein the acid is selected from the group consisting of hydrochloric acid, hydrobromic acid, sulfuric acid, nitric acid, phosphoric acid, acetic acid, propionic acid, oxalic acid, succinic acid, maleic acid, fumaric acid, methanesulfonic acid, benzenesulfonic acid, toluenesulfonic acid, citric acid, glutaric acid, citraconic acid, glutaconic acid, L-(+)-tartaric acid, D-(−)-tartaric acid, dibenzoyl-L-tartaric acid, di-p-toluoyl-L-tartaric acid, di-p-anisoyl-L-tartaric acid, (R)-(−)-α-methoxyphenyl acetic acid, L-malic acid, malonic acid, mandelic acid, (1S)-(+)-10-camphorsulfonic acid.

22. The process of claim 1, wherein the reducing agents used in step-(d) are selected from the group consisting of lithium aluminiumhydride, lithium borohydride, sodium borohydride, borane, lithium tri-ter-butoxyaluminum hydride, borane-THF complex, diisobutylaluminum hydride (DIBAL-H), and sodium bis(2-methoxyethoxy)aluminum hydride.

23. The process of claim 1, wherein the fourth solvent is selected from the group consisting of tetrahydrofuran, 2-methyl tetrahydrofuran, 1,4-dioxane, diethyl ether, diisopropyl ether, methyl tert-butyl ether, n-pentane, n-hexane, n-heptane, cyclohexane, toluene, xylene, dichloromethane, dichloroethane, chloroform, and mixtures thereof.

24. The process of claim 8, wherein [3aR-(3aα,4α,6α,6aα)]-2-[[6-amino-2,2-dimethyltetrahydro-4H-cyclopenta-1,3-dioxol-4-yl]oxy]-ethanol obtained by the process has a total purity, which includes both chemical and enantiomeric purity, of greater than about 95% as measured by HPLC.

25. The process of claim 1, further comprising the step of converting the substituted cyclopentanamine derivatives of formula II or a stereochemically isomeric form or a mixture of stereochemically isomeric forms thereof into an acid addition salt, wherein the acid addition salt is derived from a therapeutically acceptable acid selected from the group consisting of hydrochloric acid, hydrobromic acid, sulfuric acid, nitric acid, phosphoric acid, acetic acid, propionic acid, oxalic acid, succinic acid, maleic acid, fumaric acid, methanesulfonic acid, benzenesulfonic acid, toluenesulfonic acid, citric acid, glutaric acid, citraconic acid, glutaconic acid, tartaric acid, dibenzoyl-L-tartaric acid, di-p-toluoyl-L-tartaric acid, di-p-anisoyl-L-tartaric acid, (R)-(−)-α-methoxyphenyl acetic acid, L-malic acid, malonic acid, mandelic acid, and (1S)-(+)-10-camphorsulfonic acid.

26. A process for the preparation of a triazolo [4,5-d]pyrimidinecyclopentane compound, or a pharmaceutically acceptable acid addition salt thereof, comprising
providing the substituted cyclopentanamine derivative of formula II prepared according to claim 1 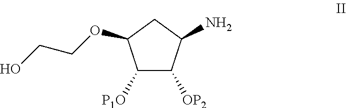
and converting the substituted cyclopentanamine derivative of formula II into the triazolo[4,5-d]pyrimidinecyclopentane compound.

27. A process for preparing pure ticagrelor comprising converting pure [3 aR-(3aα,4α,6α,6aα)]-2-[[6-amino-2,2-dimethyltetrahydro-4H-cyclopenta-1,3-dioxol-4-yl]oxy]-ethanol prepared according to claim 1 to pure ticagrelor.

28. A process for preparing a substituted cyclopentanamine derivative of formula II or a stereochemically isomeric form or a mixture of stereochemically isomeric forms thereof, comprising converting intermediate compounds of formulae V, VII and acid addition salts of VIII or a stereochemical isomer or acid addition salts thereof, prepared according to claim 1 to the substituted cyclopentanamine derivative of formula II.

29. A process for preparing ticagrelor, comprising providing the intermediate compounds of formulae V 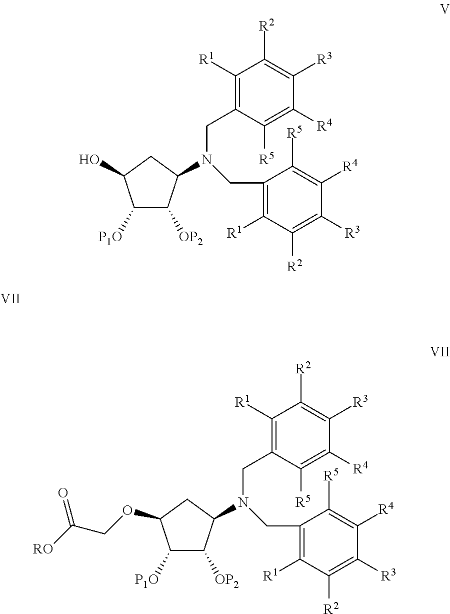
and acid addition salts of VIII 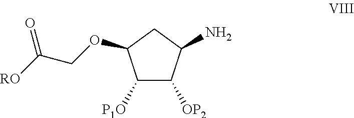
wherein P₁ and P₂ both represents hydrogen or a protecting group, or P₁ and P₂ together with the atoms to which they are attached form an alkylidene ring;
wherein R¹, R², R³, R⁴ and R⁵ are, each independently, selected from hydrogen, F, Cl, Br, I, nitro, C₁-C₃-alkyl, and C₁-C₃-alkoxy substituents; and
wherein R is C₁₋₆ straight or branched alkyl, or a benzyl group, wherein the phenyl ring of benzyl group is optionally substituted with one or more of the nitro, S(O)₂(C₁₋₄ alkyl), cyano, C₁₋₄ alkyl, C₁₋₄ alkoxy, C(O)(C₁₋₄ alkyl), N(C₁₋₆ alkyl)₂, CF₃ or OCF₃; or a stereochemical isomer or acid addition salt thereof,
and converting the intermediates to ticagrelor.
